(19) United States
(12) Patent Application Publication
(10) Pub. No.: US 2008/0200908 A1
(43) Pub. Date: Aug. 21, 2008

(54) LIGHT BEAM WAVELENGTH MIXING FOR TREATING VARIOUS DERMATOLOGIC CONDITIONS

(76) Inventor: Yacov Domankevitz, Newton, MA (US)

Correspondence Address: PROSKAUER ROSE LLP, ONE INTERNATIONAL PLACE, BOSTON, MA, 02110, US

(21) Appl. No.: 12/024,962

(22) Filed: Feb. 1, 2008

Related U.S. Application Data
(60) Provisional application No. 60/898,936, filed on Feb. 1, 2007.

(51) Int. Cl. A61B 18/18

(52) U.S. Cl. 606/9

(57) ABSTRACT

A light based treatment method and apparatus for skin photorejuvenation of a target region of skin. The treatment method uses multiple wavelength bands of light or radiation. The ratio of the energies of the wavelength bands is selected according to a skin parameter, e.g., skin type, which can be differentiated by the amount of melanin in the skin and skin condition. The invention features, in one embodiment, a safe and effective method and apparatus for treating a full range of skin types with approximately the same fluence. In some embodiments the invention can also feature a blended wavelength with a first beam of radiation with a first energy and a second beam of radiation with a second energy delivered simultaneously or sequentially. The heating of the epidermis can be maintained approximately constant regardless of skin type, leading to safe treatments and a simple protocol for all skin types.

### CROSS REFERENCE TO RELATED APPLICATION

This application claims the benefit of and priority to U.S. provisional patent application No. 60/898,936 filed Feb. 1, 2007, which is owned by the assignee of the instant application and the disclosure of which is incorporated by reference in its entirety.

### FIELD OF THE INVENTION

The invention relates generally to the an apparatus and method of performing skin photorejuvenation, particularly including treatment of vascular lesions, wrinkle, skin texture and tightening of skin using multiple wavelength bands of light.

### BACKGROUND OF THE INVENTION

Certain light sources are better suited for treating light skin, while other light sources are better suited for treating dark skin. Furthermore, some light sources can treat all skin types, but may not provide the most effective treatment. What is needed is a safe, effective treatment protocol appropriate for all skin types.

Several types of laser systems are currently being used commercially. One is the Alexandrite laser, and another is the Neodymium-YAG (Nd:YAG) laser. The laser beam of the Alexandrite laser has wavelength of about 700 nm to about 750 nm, while that of the Nd:YAG is 1,064 nm. Generally, the Alexandrite laser works better for people with light skin, while the Nd:YAG works better for people with dark skin.

The Alexandrite can also be used for people with dark skin, but the surface of the skin needs to be aggressively cooled. A very practical way to cool the skin is to spray the treatment spot with a hydrofluorocarbon refrigerant, such as 1,1,1,2-tetrafluoroethane, for a few milliseconds before the treatment pulse is delivered.

Even with aggressive cooling, dark skin may need to be tested prior to a treatment. For example, dark skin can be treated with hydroquinone for two weeks before treatments, and at the first treatment session, a practitioner can make some test spots in a normally hidden area of skin. After a two week healing period, the skin can be inspected for adverse effects before proceeding to treat a large area of skin. Clearly, even though the Alexandrite laser is a good tool for removing hair, it is less than ideal for treating dark skin.

The Nd:YAG laser, on the other hand, is an excellent device for removing hair from dark skin. The 1,064 nm wavelength of the Nd:YAG laser is less well absorbed by melanin so more of the light passes through the epidermis. The surface of the skin does not get very hot. This laser can also remove hair from people with light skin. To be effective, however, the fluence can be too high; this causes the treatment to become very painful. While the Nd:YAG laser can be used effectively for treating the dark skin, this laser is less than ideal for light skin.

It is highly desirable to treat vascular lesions such as leg and facial telangiectasias and other vascular abnormalities with light-based procedures. The Nd:YAG laser (1,064 nm) is being used to treat various vascular conditions such as leg veins, facial telangiectasias, diffused redness, etc. It is used because the 1,064 nm wavelength penetrates deeper than shorter wavelengths, and therefore the Nd:YAG laser is more effective in treating larger and deeper vessels. In addition, this wavelength is not highly absorbed by melanin, and therefore it is safer when treating darker and tanned skin. Because blood absorption at 1,064 nm is relatively low compare to the yellow-green wavelengths, higher fluence are required for effective closure of vessels. To obtain these fluences with currently available lasers, small spot in the range of 1.5 to 3 mm are often used with typical fluences ranging from 180 to 500 J/cm² depending on the spot size. When using small spot sizes, the individual vessels can be traced out. The process of tracing individual vessels tends to be slower and tedious. Larger spot size (≧6 mm) can treat larger areas faster and also penetrate deeper than smaller spots. With larger spots the user can treat larger areas without aiming at specific individual vascular lesion. With a 6 mm spot size, for example, typical fluences range between about 100-200 J/cm². Using these fluences for larger spot sizes can be very painful. In addition, with larger spots the possibility of side effects occurring can be large as well; and the risk for catastrophic full thickness ulceration and the risk profile increases. Therefore, there is a need for an improved technique for treating vascular lesions with the 1,064 nm Nd:YAG that utilizes larger spot sizes.

Furthermore, skin photorejuvenation is becoming a very popular light based procedure. One aspect of skin photorejuvenation is the improvement in skin texture including reduction of wrinkles and enlarged pores, skin tightening and skin appearance.

The improvement of skin texture is related to collagen remodeling. When the 1,064 nm laser irradiation is absorbed by blood vessels, it produces a low grade of inflammation in the vessel. Inflammatory mediators are released from the vessel into the surrounding dermis and stimulate fibroblast activity that lead to collagen remodeling and improvement in skin texture. Because blood absorption at 1,064 nm is low, higher fluences are required. A laser with 755 nm radiation is commonly used for the treatment of pigmented lesions. However, use of the Alexandrite laser can cause overheating of the epidermis if used with darker skin. This limits the scope treatments for dark skin when using the Alexandrite laser.

Therefore, there is a need for an improved skin photorejuvenation technique that allows for a single pass treatment of vascular lesions, pigmented lesions and skin photorejuvenation.

### SUMMARY OF THE INVENTION

The skin structure, in a cross-sectional view, is shown in FIG. 1. A hair shaft 2 is shown projecting from the surface of the skin 4. The structure in skin that produces a hair is called a hair follicle 12. Two areas of the hair follicle are thought to be essential for hair growth: the hair bulb 14 at the bottom of the follicle and the follicular bulge 10 located about midway along the follicle. Permanent hair removal requires that the hair follicle be damaged to the extent that the healing process cannot successfully repair the hair follicle to a functioning state. Light based hair removal methods cause this damage by heating the hair follicle. When the light enters the skin and is absorbed by the skin and hair, the light energy is converted to heat energy. An effective dosage of light raises the temperature of the hair follicle enough to permanently disable it. In other words, the hair follicle is severely burned by the light pulse.

The skin has two basic layers—the epidermis 6 and the dermis 8. The epidermis is filled with small light absorbing particles of pigment called melanin. Melanin is also found in the cortex 18 of the hair shaft and in the dermal papilla 16 of the follicle. Melanin plays an important role in the process of permanent hair removal by optical means. Below the skin is the hypodermis 10. Follicles can extend from the surface of the skin to depths ranging from 2 to 7 millimeters.

The invention features, in one embodiment, a safe and effective method and apparatus for treating a full range of skin types with approximately the same fluence. In some embodiments the invention can also feature a blended wavelength with a first beam of radiation with a first energy and a second beam of radiation with a second energy. The heating of the epidermis can be maintained approximately constant regardless of skin type, leading to safe treatments and a simple protocol for all skin types. For example, shorter wavelengths can be used to treat lighter skin, while longer wavelengths can be used to treat darker skin. Intermediate skin types can be treated with a mixture of long and short wavelength radiation in various ratios of wavelength and/or energy depending on the skin type.

Skin treatments can include treatment of vascular abnormalities and/or skin photorejuvenation. Also, a blended wavelength can be used, or in some instances, two energy beams of different wavelengths can be utilized. For vascular applications, one laser beam can modify the optical properties of blood to produce, for example, methemoglobin, a blood clot, or deoxy-hemoglobin, all of which act as a chromophore for a second beam of radiation. The second laser sees modified optical properties or created chromophores.

For skin photorejuvenation, one laser can treat one skin condition and the second laser can treat a second condition. Additionally, a single blended wavelength can be used to exploit the beneficial properties of different laser wavelengths. For skin photorejuvenation the improvement of skin texture is related to collagen remodeling. Lasers with wavelengths of 755 nm and 1,064 nm can provide different benefits when exposed to the skin. The 1,064 nm wavelength can be used for vascular lesions and the 755 nm is used for the treatment of pigmented lesions. A beam combining both wavelengths can provide effective treatment without the risk of overheating the epidermis or high fluency levels. For example, a skin photorejuvenation treatment can be performed with a single pass only. A single pass treatment can include simultaneously delivering to a target region of skin a first beam of radiation to treat a pigmentary abnormality and a second beam of radiation to treat a vascular abnormality.

In one aspect, the invention features a method of performing skin photorejuvenation. The method includes determining a skin parameter of a target region of skin and selecting a ratio of a first energy of a first beam of radiation and a second energy of a second beam of radiation based on the skin parameter. The first beam of radiation and the second beam of radiation can be delivered to the target region of skin to perform skin photorejuvenation.

In another aspect, the invention features an apparatus to perform skin photorejuvenation. The apparatus includes a first source of a first beam of radiation having a first energy and a second source of a second beam of radiation having a second energy. A controller in electrical communication with each of the first source and the second source can be used to select a ratio of the first energy and the second energy based on a skin parameter. A delivery device receives the first beam of radiation from the first source and the second beam of radiation from the second source, and can be used to deliver the first beam of radiation and the second beam of radiation to a target region of skin to perform skin photorejuvenation.

In still another aspect, the invention features a method of treating a vascular abnormality. A skin parameter of a target region of skin is determined, and a ratio of a first energy of a first beam of radiation and a second energy of a second beam of radiation is selected based on the skin parameter and/or vessel diameter, depth, and/or color. The first beam of radiation and the second beam of radiation are delivered to the target region of skin to treat the vascular abnormality in the target region, where the first beam of radiation modifies the optical properties of the target region to enhance absorption of the second beam of radiation. For example, the first beam of radiation can produce met-hemoglobin, a blood clot, and/or deoxy-hemoglobin, one or more of which act as the chromophore for the second beam of radiation. In some embodiments, the first beam of radiation provides an enhanced amount of deoxy-hemoglobin in excess of the local or naturally occurring amount of deoxy-hemoglobin.

In yet another aspect, the invention features a method of performing skin photorejuvenation. A first beam of radiation and a second beam of radiation are delivered simultaneously to rejuvenate the skin in a single pass. The first beam of radiation treats a pigmentary abnormality, and the second beam of radiation treats a vascular abnormality. The first beam of radiation can modify the optical properties of the target region to enhance absorption of the second beam of radiation. The first beam of radiation can have a wavelength of about 755 nm and the second beam of radiation can have a wavelength of about 1,064 nm. In some embodiments, a skin parameter of the target region of skin can be determined, and a ratio of the first energy of the first beam of radiation and the second energy of the second beam of radiation can be selected based on the skin parameter.

In a further aspect, the invention features a method of performing skin photorejuvenation. The method includes determining a skin parameter of a target region of skin and selecting a ratio of a first energy of a first beam of radiation and a second energy of a second beam of radiation based on the skin parameter. The first beam of radiation and the second beam of radiation can be delivered to the target region of skin to perform the skin photorejuvenation.

In another aspect, the invention features an apparatus to perform skin photorejuvenation. The apparatus includes a first source of a first beam of radiation having a first energy and a second source of a second beam of radiation having a second energy. A controller in electrical communication with each of the first source and the second source can be used to select a ratio of the first energy and the second energy such that an initial fluence of a mixture of the first beam and the second beam is independent of skin type. A delivery device receives the first beam of radiation from the first source and the second beam of radiation from the second source, and can be used to deliver the first beam of radiation and the second beam of radiation to a target region of skin to perform the skin photorejuvenation.

In still another aspect, the invention can include a means for determining a skin parameter of a target region of skin; means for selecting a ration of a first beam of radiation and a second beam of radiation based on the skin parameter; and means for delivering the first beam of radiation and the second beam of radiation to the target region of skin to treat at least one skin condition of the target region of skin.

In various embodiments, the invention can include one or more of the following features. The ratio of the first energy to the second energy can be selected such that an initial fluence of the first beam of radiation and the second beam of radiation is independent of the skin parameter. In certain embodiments, the initial fluence is between about 5 J/cm² to about 100 J/cm². In certain embodiments, the initial fluence is between about 10 J/cm² to about 50 J/cm². The first beam of radiation and the second beam of radiation can be combined into a single beam of radiation. In some embodiments, the apparatus includes a cooling device adapted to cool the target region before, during, or after the first beam of radiation or the second beam of radiation is delivered to the target region of skin.

The bandwidth of each wavelength can range from a single wavelength up to 200 nm. Each wavelength band can fall within a different range of the electromagnetic spectrum. For example, the first beam of radiation can have a wavelength between about 650 nm to about 850 nm. The second beam of radiation can have a wavelength between about 950 nm to about 1150 nm. The first beam of radiation and the second beam of radiation can be delivered substantially simultaneously. The first beam of radiation and the second beam of radiation can be combined into a single beam of radiation.

In some embodiments, the controller can select the ratio of the first energy and the second energy such that an initial fluence of the first beam of radiation and the second beam of radiation is independent of the skin parameter. The first energy can be of a first wavelength band. The second energy can be of a second wavelength band. The pulse of light energy in each wavelength band can be referred to as a band pulse. The combination of the band pulses from both wavelength bands can be referred to as a treatment pulse. The amount of energy of each wavelength band can be independently controlled. The treatment pulse can include any ratio of energies of the two wavelength bands.

The duration each beam of energy can range from 0.1 milliseconds to 500 milliseconds. A pulse can be comprised of a series of shorter pulses called sub-pulses. The duration of the treatment pulse can range from 0.1 millisecond to 500 milliseconds.

In some embodiments, the first beam of radiation has a wavelength from about 500 nm to about 1,000 nm. The first beam of radiation can have a wavelength from about 745 nm to about 760 nm. The first beam of radiation can have a wavelength from about 650 nm to about 850 nm.

In some embodiments, the second beam of radiation has a wavelength from about 750 nm to about 1,150 nm. The second beam of radiation has a wavelength from about 950 nm to about 1,150 nm. The wavelength of the second beam of radiation can be 1,064 nm.

The temporal profile of the two band pulses need not overlap. For example, a first pulse can be delivered to the skin surface, and then a second pulse of a different wavelength of light can be delivered to the skin surface. The first pulse can remove the unwanted hair, and the second pulse can remove the unwanted hair. In certain embodiments, the two band pulses are delivered substantially simultaneously to the skin surface. The two band pulses can remove unwanted hair. Skin photorejuvenation can include treating vascular lesions, pigmented lesions, wrinkles, improving skin texture, and tightening skin.

The light of each wavelength bands can irradiate and fill the same treatment spot on the skin, e.g., the region of skin irradiated by the treatment pulse. The size of the area of the treatment spot can fall within the range from 25 square millimeters to 20 square centimeters. The treatment fluence can fall within the range from 5 J/cm² to 100 J/cm².

The invention has several advantages. All skin types can be treated. A single treatment beam can be used; and a single device can be used to effect the treatment of all skin types. The new invention also allows all skin types to be treated within essentially the same fluence and/or fluence range. The ratio of the energies in the two wavelengths that comprise the treatment pulse can be adjusted according to skin type or parameter such that the recommended safe initial fluence can be essentially the same regardless of skin type. Safe effective fluences are low for all skin types and are essentially independent of skin type. The high level of pain that can be associated with the Nd:YAG when treating light skin is eliminated. The extreme heating of the epidermis that occurs when treating dark skin with the Alexandrite laser can be eliminated. Consequently, the difficult protocol is no longer necessary and the amount of required cooling can be reduced.

Other aspects and advantages of the invention will become apparent from the following drawings, detailed description, and claims, all of which illustrate the principles of the invention, by way of example only.

### BRIEF DESCRIPTION OF THE FIGURES

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a drawing of a cross-section of skin, magnified to show the basic structure of a hair follicle.

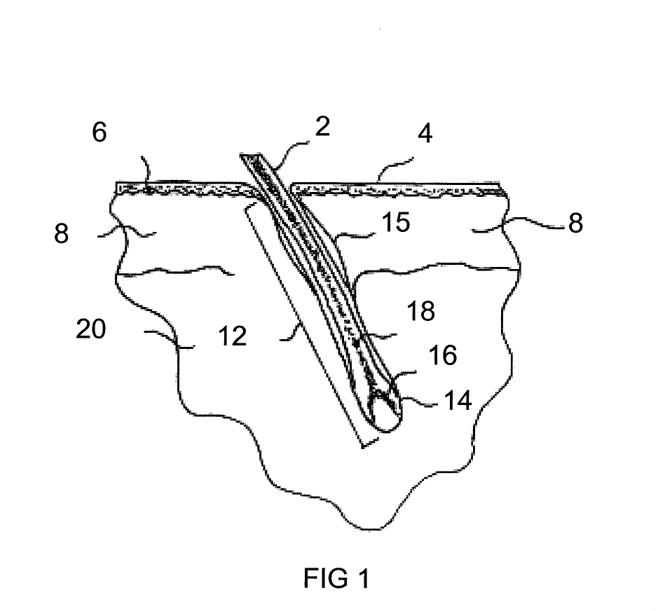

FIG. 2 is a table of skin type descriptions for the Fitzpatrick classification system.

FIG. 3 is a graph that compares the average recommended safe initial fluences by skin type for both Alexandrite and Nd:YAG hair removal laser systems.

FIG. 4 is a graph indicating the extinction coefficient of melanin over the wavelength range of 700 nm to 1100 nm.

FIG. 5 is a graph that compares the average relative recommended cooling for the same two laser systems.

FIG. 6 is a table that shows the percentages of 755 nm and 1,064 nm light that was used in calculating an initial fluence.

FIG. 7 is a table that shows the percentage ranges of 755 nm and 1,064 nm light that can be used to treat the skin

FIG. 8 is another table that shows the percentage ranges of 755 nm and 1,064 nm light that can be used in calculating an initial fluence.

FIG. 9 is a graph that shows that mixing of 750 nm and 1,064 nm in various ratios flattens out the curve for the recommended safe initial fluence for the full range of skin types.

FIG. 10 is a graph that shows that the recommended cooling also becomes nearly level across the full range of skin types.

FIG. 11 is a schematic drawing of a skin treatment device.

FIG. 12 is a schematic drawing of a skin treatment device.

FIG. 13 is a schematic drawing of a control system for skin treatment device.

FIG. 14 is a schematic drawing of a skin treatment device.

FIG. 15 is a schematic drawing of a communication network.

### DETAILED DESCRIPTION OF THE INVENTION

Wavelengths of the electromagnetic spectrum in the range from 525 nm to 1,064 nm can be used for skin treatments (e.g., hair removal because the level of optical absorption by melanin pigment is optimum or photorejuvenation including treatment of vascular lesions, wrinkle, skin texture, and tightening). Wavelengths from about 750 nm to about 1,064 nm can be particularly effective because the level of optical absorption by melanin pigment is optimum. At shorter wavelengths, most of the light is absorbed in the epidermis leading to over heating of the epidermis and/or insufficient light reaching the bulb/bulge at the bottom of the follicle. At longer wavelengths, not enough light is absorbed by the melanin pigment to be effective at safe dosage levels.

The appropriate treatment fluence depends on the wavelength of the light, the size of the treatment spot, and the amount of melanin pigment in the skin and in the hair.

Melanin is located in the epidermis, the hair bulb and the hair shaft. The concentration of melanin in the epidermis determines the shade of the skin, while the concentration of melanin in the hair determines the shade of the hair. The minimum effective fluence can be defined as the lowest fluence that can permanently remove hair at the treatment spot. The minimum effective fluence depends on the amount of melanin pigment in the hair and the hair bulb. The maximum safe fluence can be defined as the highest fluence that does not damage the epidermis. The maximum safe fluence depends on the amount of melanin pigment in the epidermis. Generally, effective hair removal is not possible when the hair is lighter than the skin because, in that case, the minimum effective fluence is higher than the maximum safe fluence.

The size of the treatment spot also affects the minimum effective fluence. The spot size can range from a fraction of one square centimeter of area to several square centimeters. With small treatment spots a significant portion of the light at the periphery of the treatment spot is scattered away from the treatment area. Since the bulbs of many of the hair follicles can be several millimeters below the surface of the skin and since the mean free path of the treatment light is about 0.8 mm in the surrounding skin tissue, much of the treatment light can be scattered away from the treatment area at the depth of the hair bulbs. The result is that the treatment fluence may need to be higher for small treatment spots than for large treatment spots. In other words, the minimum effective fluence can increase as the size of the treatment spot decreases. If the minimum effective fluence is greater than the maximum safe fluence, then a larger spot size can be used. The maximum size of the treatment spots can be limited by the amount of light energy that is available from a treatment pulse, or in some cases, pain resulting from large treatment spots.

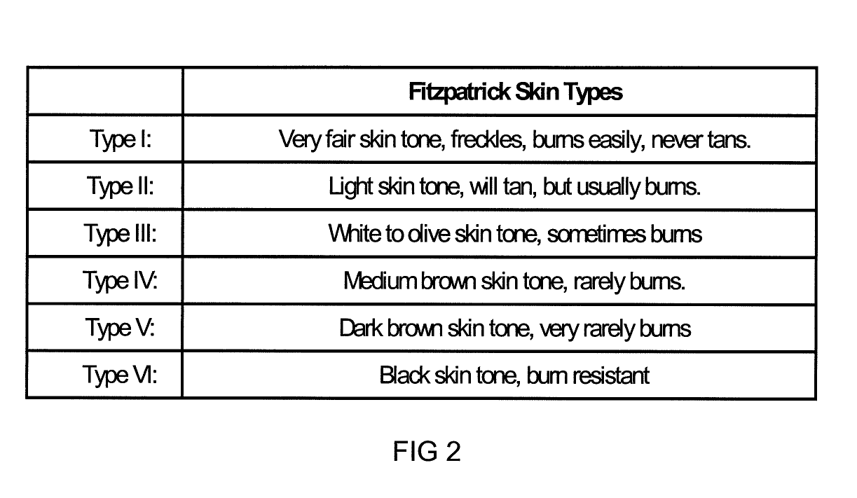

Selecting an appropriate treatment fluence can also depend on skin parameters. A classification system, called the Fitzpatrick scale, has been established for grading the darkness of skin. The scale has six degrees of darkness referred to by type I through type VI. Skin parameters can be determined by evaluation of the Fitzpatrick skin type of the patient. FIG. 2 includes descriptions of the six skin types of the Fitzpatrick scale.

Skin parameters can also be evaluated by measuring skin color with a calorimeter, or by measuring the skin pigmentation and/or erythema with a pigment/erythema meter. Skin parameter can be evaluated also for a particular segment of the treated area. Skin parameter can also be evaluated by measuring the color, pigmentation, diameter, or density of hair or any combination of these. Skin parameter can include the ratio of hair parameter to skin parameter or vice versa. Skin parameter can be also evaluated using pulsed photothermal radiometry (PPTR).

Proper treatment requires selection of an appropriate treatment fluence, but also can depend on the type of treatment that is being performed.

For vascular applications, by using a blended wavelength, one laser beam can modify the optical properties of blood. The first beam of radiation, for example can produce methemoglobin, a blood clot, or deoxy-hemoglobin, all of which act as the chromophore for a second beam of radiation. The second laser sees modified optical properties or created chromophores.

For skin photorejuvenation, one laser can treat one skin condition and the second laser can treat a second condition. Additionally, for skin photorejuvenation the improvement of skin texture is related to collagen remodeling. Laser with wavelengths of 755 nm and 1,064 nm provide different benefits when exposed to the skin. A beam combining both wavelengths provides effective treatment without the risk of overheating the epidermis or high fluency levels. For example, a skin photorejuvenation treatment can be performed with a single pass only. A single pass treatment can include delivering simultaneously to a target region of skin a first beam of radiation to treat a pigmentary abnormality and a second beam of radiation to treat a vascular abnormality to rejuvenate the skin. The 1,064 nm wavelength can be used for vascular lesions and the 755 nm wavelength can be used for the treatment of pigmented lesions.

To permanently remove hair, the hair follicle must be heated much faster than the heat can be conducted away from the follicle to the surrounding skin tissue. To effect this condition, the light is delivered in a short pulse (or short burst of sub-pulses) of light on the order of a few milliseconds in duration. Effective pulse durations range from about one millisecond to about 100 milliseconds.

During hair removal, it is advantageous to deposit as much energy deep into the hair bulb/bulge while sparing the epidermis and inducing minimal damage to the surrounding dermis. Blending wavelengths can be more effective because more energy can be deposited at the bulb/bulge. A blended laser system can be safer because less Alexandrite laser light is deposited, and the same laser effect can result. The margin of safety thus increases. With the Nd:YAG, a patient can experience pain, sometimes purpura, and it is not effective for lighter hair. With the Alexandrite, you are limited by heat absorbed by the epidermis. Even with aggressive cooling, using the Alexandrite on darker skin types can cause overheating of the epidermis. It is less than ideal for treating dark skin. The Nd:YAG is often preferred for dark skin. The Nd:YAG laser is capable of removing darker hair from lighter skin but when hair is lighter than the surrounding skin it is not generally effective because when higher energy levels are used, pain and bruising can result in certain body parts.

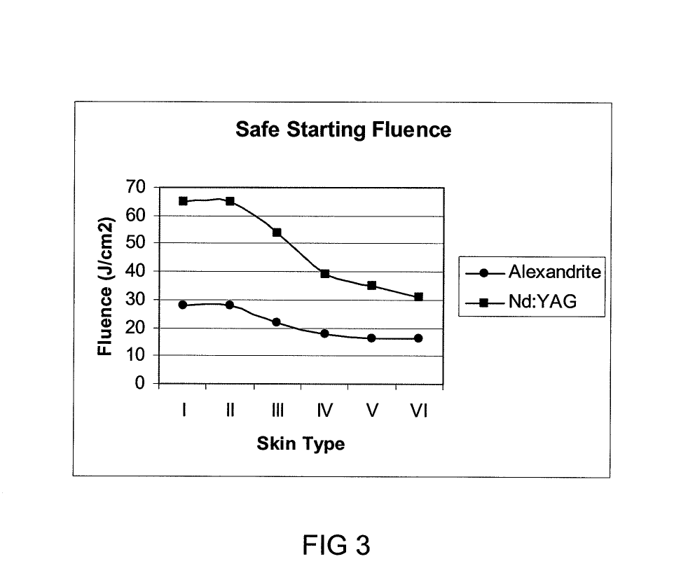

The differences between an Alexandrite laser and a Nd:YAG laser with regard to skin type can be better understood by comparing the recommended treatment dosage and epidermal cooling for the two lasers. Lists of safe but effective initial fluences and spray recommendations have been developed for the Alexandrite laser and the Nd:YAG laser, e.g., the GentleLase Alexandrite laser and the GentleYAG Nd:YAG laser available from Candela Corporation (Wayland, Mass.). FIG. 3 is a graph that compares the median suggested safe initial fluences for both lasers as a function of skin type.

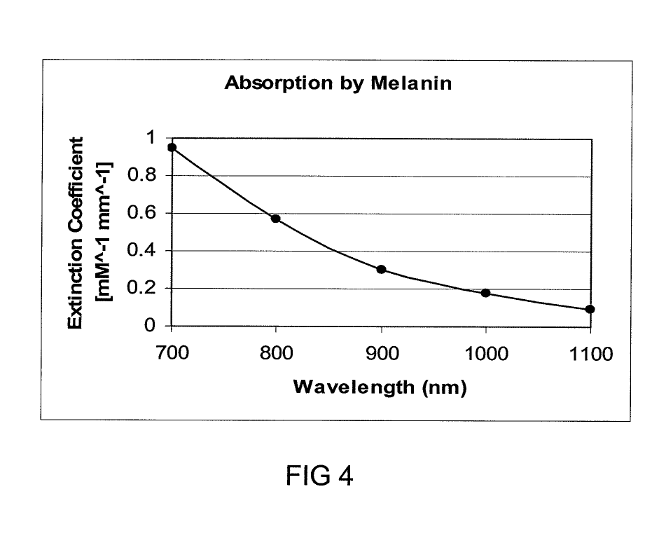

FIG. 3 illustrates that for light skin types, much higher fluences are required of the Nd:YAG laser for effective hair removal. For dark skin types, the difference between the two lasers is still large because the effective fluences for Alexandrite lasers approach the levels where epidermal damage can occur. Therefore, the suggested safe fluence for Alexandrite lasers is lower for dark skin types as compared to light skin types. The difference is explained by the amount of absorption of the treatment dose. The absorption depends on the extinction coefficient of melanin and the amount of melanin in the epidermis. The wavelength dependence of the extinction coefficient of melanin is illustrated in the graph in FIG. 4.

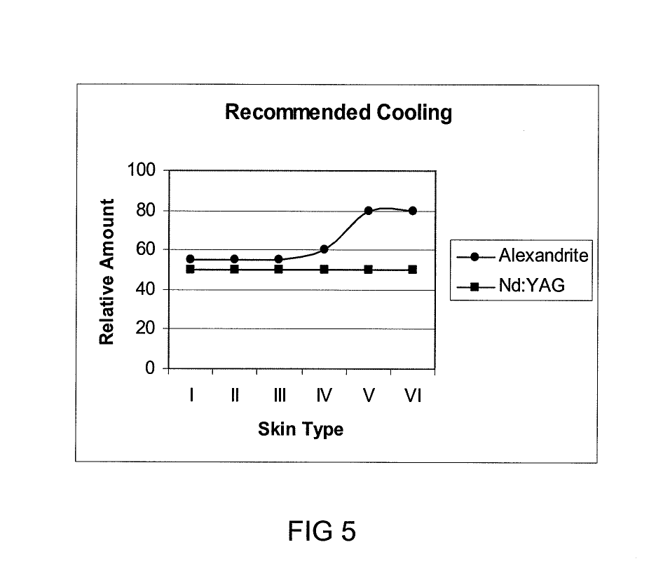

FIG. 5 is a graph that compares the relative recommended cooling for both lasers as a function of skin type. FIG. 5 shows that the cooling used with the Alexandrite laser and the Nd:YAG laser is nearly the same for the lighter skin types. Here, the epidermis is only moderately heated by the two lasers. For the darker skin types, the epidermal heating by the Alexandrite laser is significantly greater, and therefore much more cooling is recommended.

A hair follicle can be permanently disabled when the follicular bulge and the hair bulb are both sufficiently damaged to prevent the healing process from restoring the functions of these two structures. Monte Carlo modeling of hair removal, first with the Alexandrite laser and then with the Nd:YAG laser, indicates that the bulge is the more difficult structure to heat. That is, if the fluence is high enough to permanently disable the bulge, then the hair bulb can be destroyed, resulting in permanent hair removal. The bulge typically does not contain melanin, and is not well heated by the light dose. Instead, it is heated by the conduction of heat from the hair shaft. The hair shaft can be heated to raise the temperature of the bulge above the damage threshold. The conclusion from the modeling is that both lasers can heat the bulge enough to cause permanent hair removal. This suggests that the recommended safe initial fluence is the fluence that raises the bulge just enough to cause lasting damage. The rise in temperature is proportional to the fluence. Therefore, the fluence of each laser can be adjusted to achieve the desired fractional thermal contribution when using both lasers at the same time.

A system that adjusts the fractional thermal contribution of one or more lasers can combine two treatment beams to remove unwanted hair or to treat a skin condition. The system can include a first source of a first beam of radiation and a second source of a second beam of radiation. The energies of the beams can be selected based on a skin parameter. A delivery device can deliver the beams of radiation and the second beam of radiation to a target region of skin to remove at least one unwanted hair. The system allows a full range of skin types to be treated with approximately the same fluences. This makes the treatments tolerable for patients of all skin types and can increase the safety margin for effective hair removal and skin treatments.

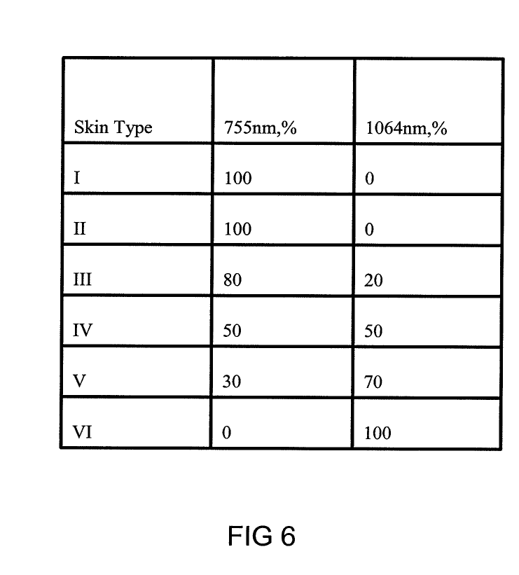

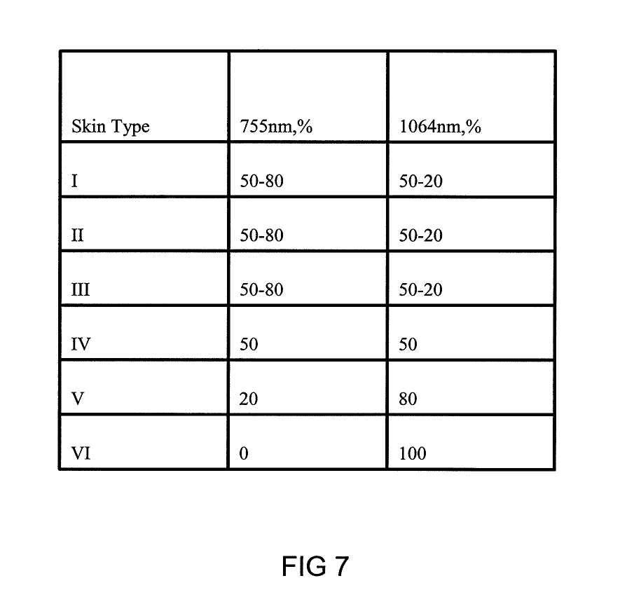

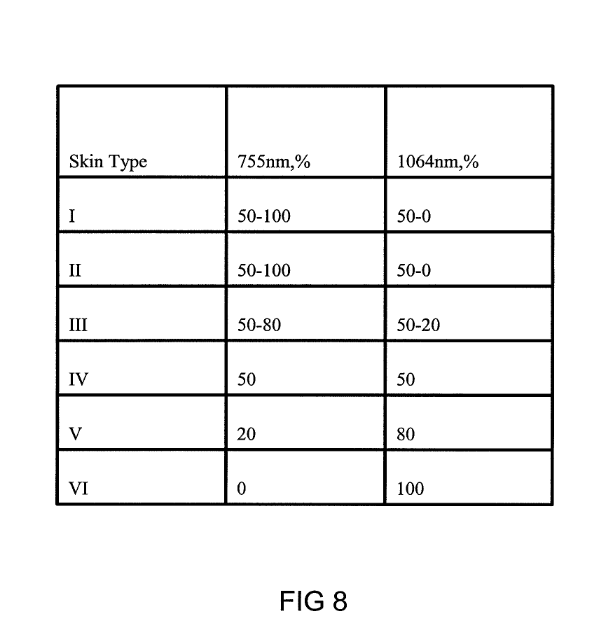

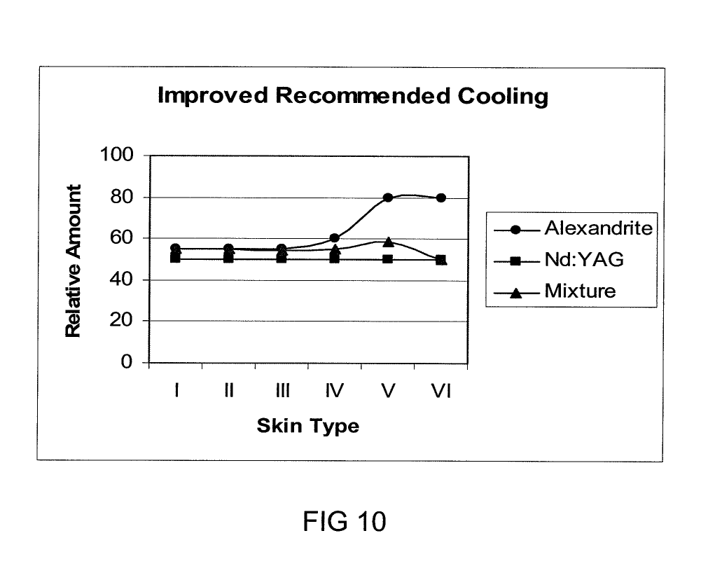

FIG. 6 shows the percentage of 755 nm and 1,064 nm light that can be used in calculating an initial fluence of a system that combines two or more energy beams of radiation. FIG. 6 gives the percentages of 755 nm and 1,064 nm that were used in calculating the suggested initial fluence for the “Mixture” curve in FIG. 10. FIG. 6 also shows that each light source can supply between 0% and 100% of the total energy needed to supply the suggested initial fluence. FIGS. 7 and 8 shows the percentage ranges of 755 nm and 1,064 nm that can be used to treat the skin.

Other ratios can be used to provide treatment. For example, since 1,064 nm is not scattered as much as 755 nm, increasing the relative amount of 1,064 nm can improve the effectiveness of small spot sizes. As another example, since 1,064 nm has deeper penetration, increasing the ration of 1,064 nm can improve the effectiveness when treating hair that is relatively light compared to the skin. As another example, one might set the fluence of one of the wavelength bands to a level that is safe regardless of skin types and then add fluence of the other wavelength band to bring the total fluence to a safe and effective level.

The same condition similarly affect the heating of the epidermis. The recommended cooling is an indication of the amount of epidermal heating. Mixing the wavelengths in the same ratios as indicated in FIG. 6 and calculating the cooling requirements provided for the recommended cooling parameters shown in FIG. 7 reduces excessive heating of dark skin by the Alexandrite laser. This facilitates treatment of dark skin with an Alexandrite laser system. In one embodiment, both a 755 nm wavelength energy beam and the 1,064 nm wavelength energy beam can be delivered simultaneously or sequentially to the skin. For example, the 755 nm heats up the blood in the vessels to a temperature enough to induce methemoglobin within the vessel blood. Because the blood absorption at 755 nm is about twice the absorption at 1,064 nm and because the absorption of methemoglobin at 755 nm is almost 5 times of that at 1,064 nm, the total energy used for the treatment of vascular lesions is less than the 1,064 nm wavelength by itself. Reducing the energy used can significantly reduce pain and allow for a safer treatment. Methemoglobin can be generated when the 755 nm and 1,064 nm irradiations are being delivered at the same time or sequentially. The use of blended wave is not limited to the production of methemoglobin only but can take advantage of other optical properties modifications such as clot formation and/or the production of deoxy-hemoglobin. In one aspect, the 755 nm is irradiated simultaneously with the 1,064 nm. In another aspect, the 755 nm is irradiated first and before the 1,064 nm. In another aspect, the 1,064 nm is irradiated before the 755 nm wavelength.

Similarly, the mixing or blending of the two lasers or their use in sequence can reduce the total amount of energy used during the treatment of skin texture and tightening. For example, mixing or blending the 1,064 nm and the 755 nm wavelengths allows for a single pass skin photorejuvenation. The 755 nm treats the pigmented lesions and the 1,064 nm treats the vascular lesions. In addition, the 755 nm treats the pigmented lesions and because it penetrates deeper than the lesions, it also modifies the optical properties of the vessels to be treated and allows these vessels to be treated with less 1,064 nm energy. Similarly, less total energy can be used for treating wrinkles and for tightening.

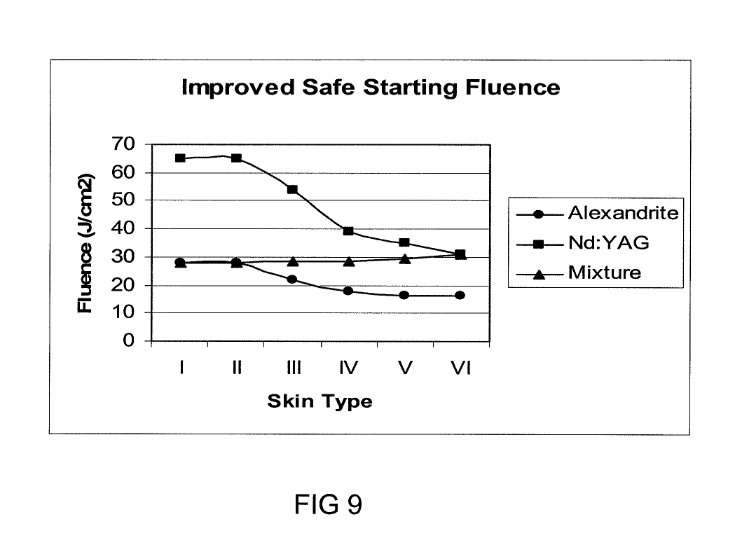

The graphs in FIGS. 9 and 10 further illustrate that low treatment fluence and low epidermal heating can be provided for all skin types. Low treatment fluence can make the treatment less painful, especially for people with light skin types. Low epidermal heating can make the treatment easier and safer, especially for people with dark skin types. The “Mixture” curve in the graph in FIG. 9 further suggests that the safe initial fluence can be selected essentially independent of skin type when the fractions of the two wavelengths are adjusted for skin type. FIG. 10 shows recommended cooling can also be selected largely independent of the skin type when a “Mixture” treatment is used.

An exemplary embodiment for skin type IV can be determined from FIG. 10. If half of the temperature rise in the bulge is caused by the 755 nm radiation, then the fluence of the 755 nm radiation can be cut in half to about 9 J/cm². Likewise, the 1,064 nm radiation is changed to give 20 J/cm². Then irradiating with the two beams at the same time, a total fluence of about 29J/cm² heats the bulge to the full effective temperature.

In practice, the invention can include at least one of the following steps: providing an apparatus as described above, selecting the treatment spot size, determining the skin type of the patient, selecting the energies for each band pulse, selecting the location of the treatment spot, optionally cooling the treatment, irradiating the treatment spot, and then repeating the locating for a new treatment spot, optionally cooling and irradiating the treatment spot, until the entire area to be treated has been treated. The cooling and irradiating may be concurrent.

A treatment can include at least one of the following steps: providing an apparatus as described above, selecting the treatment spot size, determining the skin type of the patient, selecting a ratio of the energies for each band pulse based on a skin parameter, selecting the location of a treatment zone, cooling the treatment zone, providing radiation to the treatment zone, and observing the results. A treatment test spot can be selected and irradiated. If necessary, the total fluence can be adjusted and additional test spots can be applied as above in order to find a safe and effective treatment fluence.

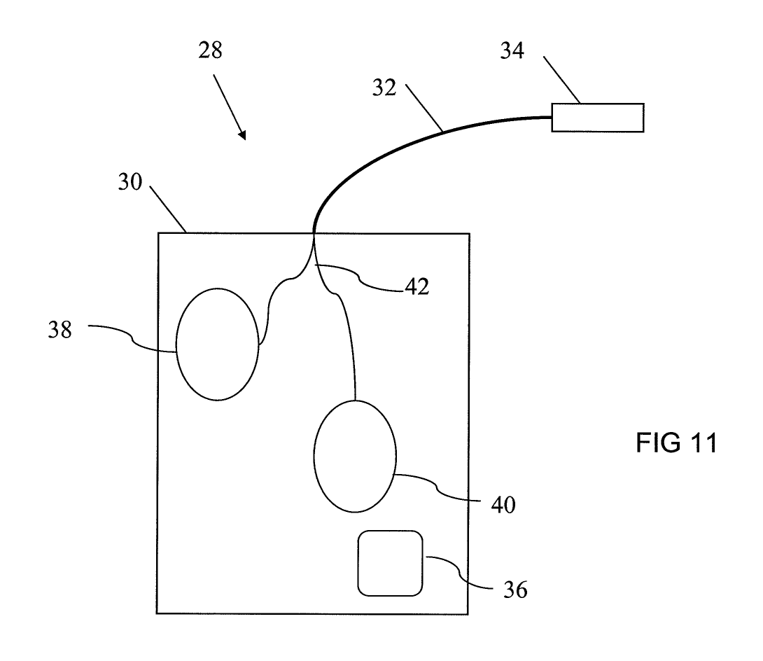

FIG. 11 shows a schematic drawing of an embodiment of a device 28 that can mix wavelengths of two treatment beams. The device 28 includes a cabinet 30, a delivery system 32 and a handpiece 34. The cabinet 30 houses a control system 36, a first radiation source 38, and a second radiation source 40. Output from the first radiation source 38 and the second radiation source 40 can be combined using an optical system 42 and coupled to the delivery system 32.

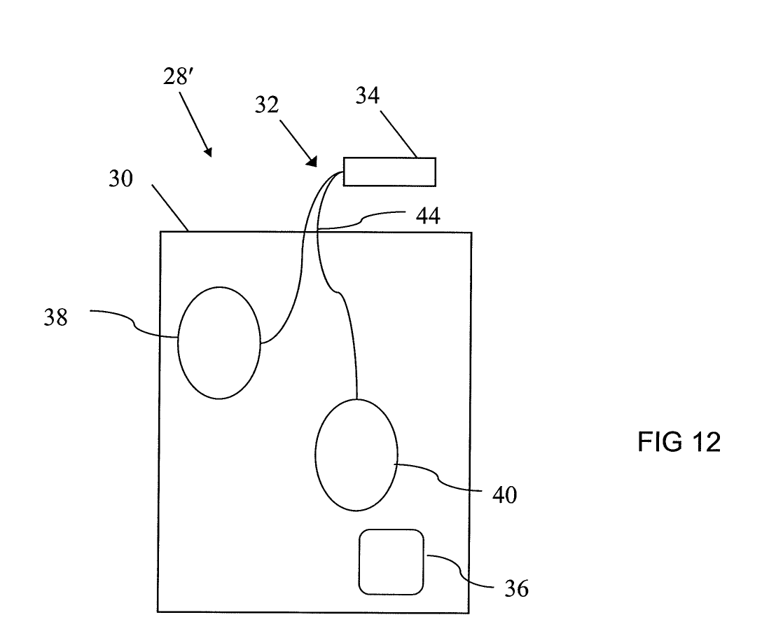

FIG. 12 shows a schematic drawing of an embodiment of a device 28′ that can combine output from the first radiation source 38 and the second radiation source 40 at the handpiece 34. The delivery system 32 can include two optical fibers 44, each directing radiation from one of the radiation sources to the handpiece 34.

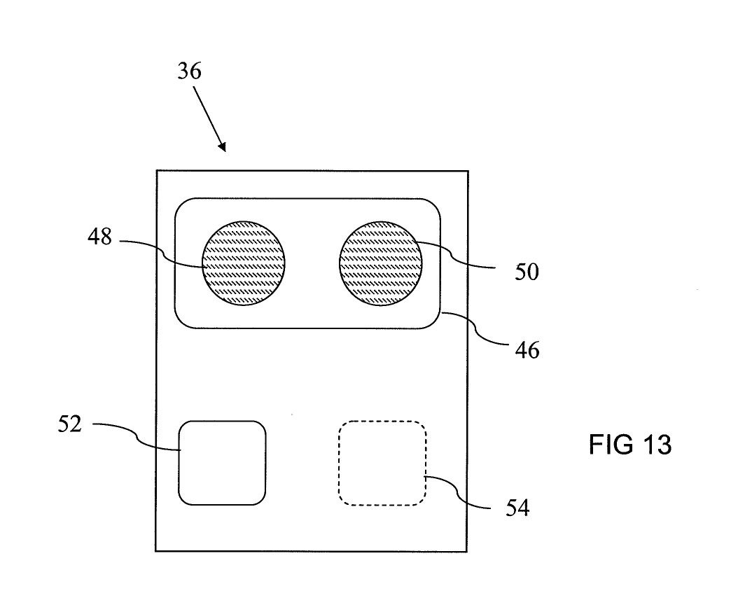

FIG. 13 shows an embodiment of the control system 36, which can include a user interface 46 having a first control 48 for the energy of the device 28 and a second control 50 for the ratio of output of the first radiation source 38 and the second radiation source 40. The control system 36 can include a processing unit 52 and optionally include a memory device 54. The user interface 46 and/or the processing unit 52 can be used to modulate treatment parameters and/or properties of the emitted the electromagnetic radiation, including one or more of the following: the portion of the electromagnetic spectrum used, pulse-width, pulse-shape, application time, power, and/or fluence. In some embodiments, the one or more treatment parameters can include the duration, degree, and/or other parameters of cooling used in conjunction with the electromagnetic radiation.

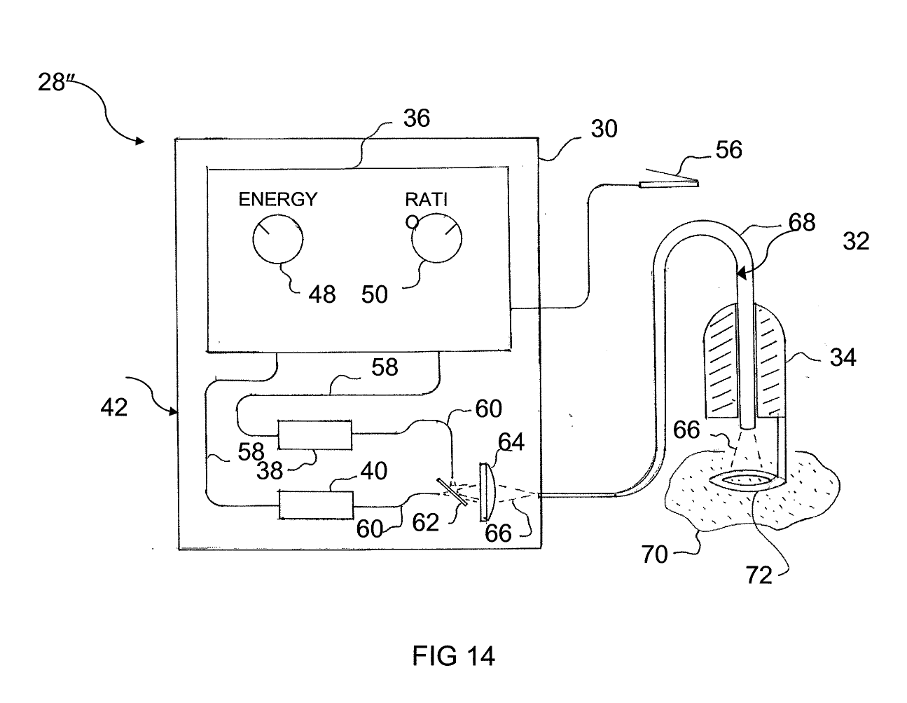

FIG. 14 shows a schematic drawing of another embodiment of a device 28″ that can mix wavelengths of two treatment beams. The device 28″ includes a cabinet 30, a delivery system 32 and a handpiece 34. The cabinet 30 houses a control system 36, a first radiation source 38, and a second radiation source 40. Output from the first radiation source 38 and the second radiation source 40 can be combined using an optical system 42 and coupled to the delivery system 32. The control system 36 includes a first control 48 for the energy of the device 28″ and a second control 50 for the ratio of output of the first radiation source 38 and the second radiation source 40. A trigger 56 (e.g., a foot switch, a hand switch, or a trigger initiated by a processor 52 as shown in FIG. 13) can be used to trigger the radiation sources.

The control system 36 delivers control signals to the first radiation source 38 and the second radiation source 40 via signal carriers 58. In certain embodiments, the signal carrier 58 is a wireless connection. The optical system 42 includes waveguides 60 (e.g., an optical fiber) to deliver output from the first radiation source 38 and the second radiation source 40 to a dichroic beam combiner 62, which directs mixed radiation to a lens 64. The mixed radiation 66 is coupled to the delivery system 32, which can include a waveguide 68 (e.g., single 1 mm diameter optical fiber). The handpiece 34 directed the mixed radiation beam 66 to a target region of skin 70. The handpiece 34 can include a spacer 72 to space the handpiece 34 from the surface of the skin 70. The handpiece 34 can include one or more lens to image the treatment beam on the target region. In one embodiment, two laser handpieces can be used—one for each beam of radiation.

In various embodiments, one or both of the radiation sources 38, 40 can be a coherent source (e.g., a laser) or an incoherent source (e.g., a lamp, a flashlamp, an incandescent lamp, a light emitting diode, an intense pulsed light system, or a fluorescent pulsed light system). Lasers include solid state laser, diode lasers, diode laser arrays, fiber coupled diode laser arrays, optically combined diode laser arrays, and/or high power semiconductor lasers. Suitable diode lasers include a 200 W, 780 nm, fiber-coupled diode laser and a 200 W, 980 nm, fiber-coupled diode laser. Suitable solid state lasers include a 755 nm alexandrite laser and a 1,064 nm Nd:YAG laser. The 532 nm output of a Nd:YAG laser can be combined with the 1,064 nm output of a Nd:YAG laser.

Incoherent sources include fluorescent pulsed light (FPL) systems. FPL technology can utilize laser-dye impregnated polymer filters to convert unwanted energy from a xenon flashlamp into wavelengths that enhance the effectiveness of the intended applications. FPL technologies can be more energy efficient and can generate significantly less heat than comparative IPL systems. A FPL system can be adapted to operate at a selected wavelength or band of wavelengths by changing filters or hand pieces.

The hand piece 34 can modulate the temperature in a region of biological tissue and/or minimize unwanted thermal injury to untargeted biological tissue. By cooling only a region of the target region or by cooling different regions of the target region to different extents, the degree of thermal injury of regions of the target region can be controlled. For example, the hand piece 34 can cool the biological tissue before, during, or after delivery of radiation, or a combination of the aforementioned. Cooling can include contact conduction cooling, evaporative spray cooling, convective air flow cooling, or a combination of the aforementioned. In one embodiment, the hand piece 34 includes a biological tissue contacting portion that can contact a region of biological tissue. The biological tissue contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window to cool the biological tissue.

A spray cooling device can use cryogen, water, or air as a coolant. In one embodiment, a dynamic cooling device (e.g., a DCD available from Candela Corporation) can cool the biological tissue. For example, the delivery system 32 can include tubing for delivering a cooling fluid to the hand piece 34. The tubing can be connected to a container of a low boiling point fluid, and the hand piece 34 can include a valve for delivering a spurt of the fluid to the biological tissue. Heat can be extracted from the biological tissue by evaporative cooling of the low boiling point fluid. In one embodiment, the fluid is a non-toxic substance with high vapor pressure at normal body temperature, such as a Freon or tetrafluoroethane.

The control system 36 can receive input from the practitioner regarding the patient's skin type, the amount of cooling desired, the treatment spot size, the fluence, the pulse duration, and/or treatment pulse repetition rate. A control signal can initiate a single treatment pulse or a series of treatment pulses emitted at a selected pulse repetition rate. The control system 36 can control the timing of the initiation of the spray, the duration of the spray, a short delay between the end of the spray and the initiation of laser output, and the delay between the initiations of the two radiation sources if sequential pulsing is desired. Additional spray can be administered to the treatment spot between the pulses or during pulses. If the selected pulse duration is longer than the maximum pulse duration of a single pulse, then the control system initiates a series of shorter sub-pulses, and controls the delay between initiation of each sub-pulse so that the envelope of the sub-pulses equals the selected pulse duration. The control system can also control the peak power output of both lasers, and the duration or energy from each pulse or sub-pulse.

In various embodiments, one of the beams of radiation has a wavelength between about 400 nm and about 2,600 nm, although longer and shorter wavelengths can be used depending on the application. In some embodiments, the wavelength can be from about 500 nm to about 1,200 nm. The wavelength of the first beam of radiation can be from about 500 nm to about 1,000 nm. The wavelength of the first beam of radiation can be from about 745 nm to 760 nm. The wavelength of the first beam of radiation can be about 755 nm. The wavelength of the first beam of radiation can be about 532 nm. The wavelength of the first beam of radiation can be about 780 nm. The wavelength of the second beam of radiation can be from about 600 nm to about 1,200 nm. The wavelength of the second beam of radiation can be from about 750 nm to about 1,150 nm. The wavelength of the second beam of radiation can be from about 950 nm to about 1,150 nm. The wavelength of the second beam of radiation can be 980 nm. The wavelength of the second beam of radiation can be 1,064 nm. One or more of the wavelengths used can be within a range of wavelengths that can be transmitted to the target region of skin to treat or remove a hair follicle.

In certain embodiments, the bandwidth of a wavelength for a radiation source can range from a single wavelength up to a 200 nm band of wavelengths. Each wavelength band can fall within a different range of the electromagnetic spectrum. For example, the first beam of radiation can have a wavelength or wavelength band from about 650 nm to about 850 nm. The second beam of radiation can have a wavelength or wavelength band from about 950 nm to about 1,150 nm.

The temporal profile of the two band pulses need not overlap. For example, a first pulse can be delivered to the skin surface, and then a second pulse of a different wavelength of light can be delivered to the skin surface. The first pulse can remove the unwanted hair, and the second pulse can remove the unwanted hair. In certain embodiments, the two band pulses are delivered substantially simultaneously to the skin surface. The two band pulses can remove unwanted hair.

The light of each radiation beam can irradiate and fill the same treatment spot on the skin, e.g., the region of skin irradiated by the treatment pulse. The size of the area of the treatment spot can fall within the range from about 25 mm² to about 20 cm², although larger and smaller treatment zones can be used depending on the application. In various embodiments, the beam of radiation can have a spot size from about 0.5 mm to about 25 mm, although larger and smaller spot sizes can be used depending on the application.

In various embodiments, the treatment can deliver a fluence from about 1 J/cm² to about 500 J/cm², although higher and lower fluences can be used depending on the application. In some embodiments, the fluence can be from about 5 J/cm² to about 150 J/cm². In some embodiment, the fluence is from about 5 J/cm² to about 100 J/cm². In some embodiment, the fluence is from about 10 J/cm² to about 50 J/cm². In some embodiment, the fluence is from about 25 J/cm² to about 35 J/cm². The fluence can be about 25 J/cm², about 35 J/cm², or about 50 J/cm². In various embodiments, a treatment exposes target tissue to a cumulative fluence greater than the fluence of the individual beams of radiation.

In various embodiments, the pulse duration can be from about 10 μs to about 30 s, although larger and smaller pulse durations can be used depending on the application. In various embodiments, the pulse duration is about 0.1 ms to 500 ms. The pulse duration can be from about 1 ms to about 100 ms. The pulse duration can be less than 5 ms. The pulse duration can be from about 1 ms to about 3 ms. The pulse duration can be from about 1 ms to about 2 ms. The pulse duration can be 3 ms. A pulse can be comprised of a series of shorter pulses called sub-pulses.

In various embodiments, the parameters of the radiation can be selected to deliver the radiation to a predetermined depth. In some embodiments, the beam of radiation can be delivered to the target region about 0.5 mm to about 10 mm below an exposed surface of the skin, although shallower or deeper depths can be selected depending on the application.

In various embodiments, the tissue can be heated to a temperature of between about 50° C. and about 80° C., although higher and lower temperatures can be used depending on the application. In one embodiment, the temperature is between about 55° C. and about 70° C.

The hand piece 34 that delivers the blended or mixed wavelength can have a switch that enables the delivery of each wavelength. For example, when treating a face mixed with pigmented lesions (brown) and vascular (reds) lesions it might be advantageous to use the 755 nm only when there is an area with pigmented lesions only. When treating an area with vascular lesion only, the user can select either the 1,064 nm only or the blended 1,064/755 nm. In certain embodiments, the hand piece 34 includes an evacuation chamber so that the skin can be compressed before, during and after treatment. The evacuation chamber is provided with an essentially rigid interface element larger than a threshold surface area through which radiation can be administered to a target skin region. One or more walls of the evacuation chamber can contact the skin region, and the skin can be drawn against the interface element when negative pressure (e.g., vacuum) is applied. Compression of the target region of skin can remove unwanted chromophores (e.g., blood) from the target region to increase the efficiency of radiation absorption in the target region. Furthermore, compression can inhibit the transmission of a pain signal generated by pain receptors located within the target skin region. Suitable evacuation chambers and systems for generating a negative pressure are described in one or more of the following U.S. patent applications, the entire disclosure of each herein incorporated by reference in its entirety: Ser. Nos. 11/498,456; 11/401,674; and 11/057,542.

Materials that are transparent at the treatment wavelength(s) can be in placed over the treatment spot during the treatment macro-pulse. These materials can also compress the skin to remove blood and improve the transparency of the skin and hypodermis, or compress the skin to thin the skin and hypodermis and thereby reduce the path length of the radiation within the target tissue so that the amount of light that is absorbed by the hair, the hair bulge, and/or the hair bulb is increased. An evacuation device can be combined with, for example, contact cooling to compress the skin and reduce pain.

The optical system 42 need not be used to combine beams. For example, two flash lamps can be located in an irradiation module, each one having it own spectral filter. The light from the two sources can be allowed to overlap at the treatment zone. The output of pigtailed diode lasers and/or LEDs can be combined by configuring them to overlap at the treatment zone. Furthermore, the optical system 42 can include a polarization optic to combine beams. Polarized beams can be combined with polarization sensitive prisms. Laser and LED sources can be combined by focusing the beams into the separate ends of a bifurcated fiber optic bundle. Polarized or non-polarized beams can be combined with a dichroic beam combiner.

Although the embodiments shown in the figures use two radiation sources, the invention is not limited to embodiments having two different radiation sources. Other embodiments can use three or more different radiation sources to treat the skin condition. Furthermore, a single radiation source can generate two wavelengths or wavelength bands suitable for treatment. The total energy supplied by the one or more radiation sources can equal 100% of the total energy needed to successfully treat the skin condition.

In various embodiments, the control system 36 of the device 28 can send and/or receive information to and from a remote site through a network. For example, treatment parameters can be stored remotely and accessed when a particular reaction is identified by a user. This permits an outside agency to change, update, or add treatment parameters as new parameters are determined, e.g., by academic research or clinical studies.

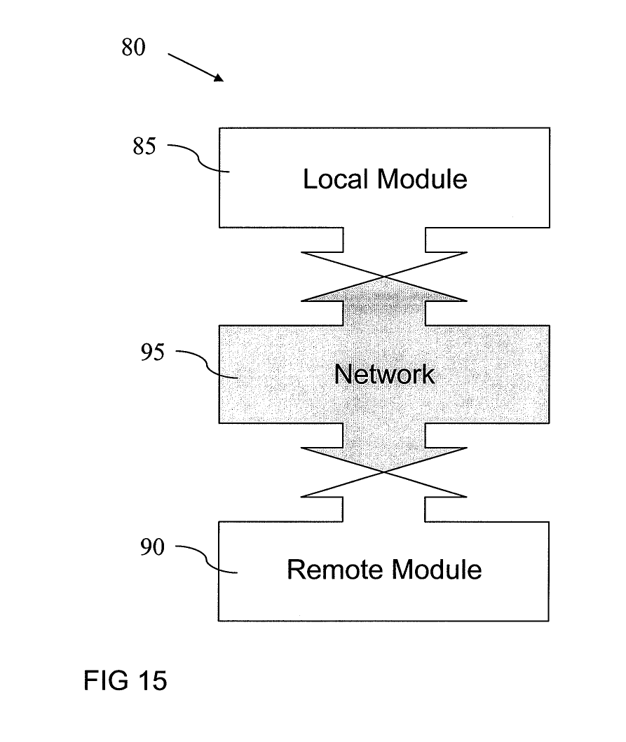

FIG. 15 shows an exemplary network system 80 including a local module 85 and a remote module 90, which are in communication through a communication network 95. The local module 85 is configured to provide treatments using the technology. In various embodiments, the local module 85 can include one or more computers, servers, firewalls, databases, or other network devices to process, send, and/or receive information through the communication network 95. The remote module 90 can include one or more computers, servers, firewalls, databases, or other network devices to process, send, and/or receive information through the communication network 95. The communication network 95 can be a private company network, for example an intranet, or a public network, for example the internet. The communication network 95 can be wired or wireless.

In various embodiments the local module 85 can transmit information to the remote module 90. For example, the local module 85 can transmit information relating to the biological tissue to be treated and/or information relating to at least one reaction between the biological tissue and the electromagnetic radiation. Based upon the information, the remote module 90 can provide one or more treatment parameters based upon the information provided. The remote module 90 can calculate treatment parameters and/or retrieve treatment parameters from a database. In some embodiments, the remote module 90 can collect, store, and/or analyze information from multiple treatments by a user and/or multiple users. Furthermore, the local module 85 can receive treatment parameters from the remote module 90 and provide a treatment. A user can edit treatment parameters at a remote module 90 using a website (e.g., of the treatment provider) to access a database containing the treatment parameters. Furthermore, a patient's response to a treatment can be recorded at the local module 85 and communicated to and stored at the remote module 90.

The above-described techniques can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps can be performed by one or more programmable processors executing a computer program to perform functions of the technology by operating on input data and generating output. Method steps can also be performed by, and apparatus can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (IC), FPGA or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (PBX). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks. Communication networks can also all or a portion of the PSTN, for example, a portion owned by a specific carrier.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

1. A method of performing skin photorejuvenation:
determining a skin parameter of a target region of skin;
selecting a ratio of a first energy of a first beam of radiation and a second energy of a second beam of radiation based on the skin parameter; and
delivering the first beam of radiation and the second beam of radiation to the target region of skin.

2. The method of claim 1 further comprising determining the ratio of the first energy to the second energy such that an initial fluence of the first beam of radiation and the second beam of radiation is independent of the skin parameter.

3. The method of claim 1 further comprising combining the first beam of radiation and the second beam of radiation into a single beam of radiation.

4. The method of claim 1 further comprising cooling the skin before, during, or after delivering the first beam of radiation and the second beam of radiation.

5. The method of claim 2 wherein the initial fluence is between about 10 J/cm² to about 50 J/cm².

6. The method of claim 1 wherein skin photorejuvenation comprises treating vascular lesions, pigmented lesions, wrinkles, improving skin texture, and tightening skin.

7. An apparatus for performing skin photorejuvenation comprising:
a first source of a first beam of radiation having a first energy;
a second source of a second beam of radiation having a second energy;
a controller in electrical communication with each of the first source and the second source, the controller selecting a ratio of the first energy and the second energy based on a skin parameter of a target region of skin; and
a delivery device receiving the first beam of radiation from the first source and the second beam of radiation from the second source to deliver the first beam of radiation and the second beam of radiation to the target region of skin.

8. The apparatus of claim 7 further comprising a cooling device adapted to cool the target region before, during, or after the first beam of radiation or the second beam of radiation is delivered to the target region of skin.

9. The apparatus of claim 7 wherein the first beam of radiation has a wavelength between about 650 nm to about 850 nm.

10. The apparatus of claim 7 wherein the second beam of radiation has a wavelength between about 950 nm to about 1150 nm.

11. The apparatus of claim 7 wherein the controller selects the ratio of the first energy and the second energy such that an initial fluence of the first beam of radiation and the second beam of radiation is independent of the skin parameter.

12. The apparatus of claim 7 wherein the controller selects the initial fluence between about 10 J/cm² to about 50 J/cm².

13. A method of treating a vascular abnormality, comprising:
determining a skin parameter of a target region of skin;
selecting a ratio of a first energy of a first beam of radiation and a second energy of a second beam of radiation based on the skin parameter; and
delivering the first beam of radiation and the second beam of radiation to the target region of skin to treat the vascular abnormality in the target region, where the first beam of radiation modifies the optical properties of the target region to enhance absorption of the second beam of radiation.

14. The method of claim 13 further comprising determining the ratio of the first energy to the second energy such that an initial fluence of the first beam of radiation and the second beam of radiation is independent of the skin parameter.

15. The method of claim 13 wherein the first beam of radiation produces at least one of methemoglobin, a blood clot, and deoxy-hemoglobin, one or more of which act as the chromophore for the second beam of radiation.

16. The method of claim 13 further comprising delivering the first beam of radiation and the second beam of radiation substantially simultaneously.

17. A method of performing skin rejuvenation, comprising:
delivering simultaneously to a target region of skin a first beam of radiation to treat a pigmentary abnormality and a second beam of radiation to treat a vascular abnormality to rejuvenate the skin in a single pass.

18. The method of claim 17 further comprising delivering the first beam of radiation and the second beam of radiation substantially simultaneously.

19. The method of claim 17 wherein the first beam of radiation modifies the optical properties of the target region to enhance absorption of the second beam of radiation.

20. The method of claim 17 wherein the first beam of radiation has a wavelength of about 755 nm and the second beam of radiation has a wavelength of about 1,064 nm.

21. The method of claim 17 further comprising:
determining a skin parameter of a target region of skin; and
selecting a ratio of the first energy of the first beam of radiation and the second energy of the second beam of radiation based on the skin parameter.

22. An apparatus for skin rejuvenation comprising:
a first source of a first beam of radiation having a first energy;
a second source of a second beam of radiation having a second energy;
a controller in electrical communication with each of the first source and the second source, the controller selecting a ratio of the first energy and the second energy such that an initial fluence of a mixture of the first beam of radiation and the second beam of radiation is independent of skin type; and
a delivery device receiving the first beam of radiation from the first source and the second beam of radiation from the second source to deliver the mixture of the first beam of radiation and the second beam of radiation to a target region of skin to treat at least one skin condition.

23. An apparatus for skin rejuvenation comprising;
means for determining a skin parameter of a target region of skin;
means for selecting a ratio of a first energy of a first beam of radiation and a second energy of a beam of radiation based on the skin parameter; and
means for delivering the first beam of radiation and the second beam of radiation to the target region of skin to treat at least one skin condition of the target region of skin.

24. The apparatus of claim 23 wherein the means for selecting the ratio of the first energy and the second energy selects that ratio such that an initial fluence of the first beam of radiation and the second beam of radiation is independent of the skin parameter.
